(19) United States
(12) Patent Application Publication
(10) Pub. No.: US 2025/0295678 A1
(43) Pub. Date: Sep. 25, 2025

(54) ULTRA LOW DOSAGE ZOLEDRONIC ACID FOR TREATMENT OF RETINAL DISEASE

(71) Applicant: The Regents of the University of California, Oakland, CA (US)

(72) Inventors: Aparna Lakkaraju, Oakland, CA (US); Li Xuan Tan, Oakland, CA (US); Colin Germer, Oakland, CA (US)

(21) Appl. No.: 18/863,537

(22) PCT Filed: May 10, 2023
(86) PCT No.: PCT/US2023/021675

§ 371 Date: Nov. 6, 2024

Related U.S. Application Data
(60) Provisional application No. 63/340,435, filed on May 10, 2022.
(60) Provisional application No. 63/500,448, filed on May 5, 2023.

(51) Int. Cl. A61K 31/675; A61K 31/4468; A61P 27/02

(52) U.S. Cl. CPC A61K 31/675; A61K 31/4468; A61P 27/02

(57) ABSTRACT

Provided herein are method of treating retinal disease using a bisphosphonate acid sphingomyelinase (ASM) and farnesyl diphosphate synthase (FDPS) inhibitor, such as zoledronic acid, at low doses.

### CROSS-REFERENCES TO RELATED APPLICATIONS

This application claims priority benefit of U.S. Provisional Application No. 63/340,435, filed May 10, 2022 and U.S. Provisional Application No. 63/500,448, filed May 5, 2023, each of which is herein incorporated by reference for all purposes.

### BACKGROUND OF THE INVENTION

The retinal pigmented epithelium (RPE) is vital component of the eye. The RPE is made up of a flat mosaic of hexagonal cells, tightly bound at their junctions. The RPE is adjacent, on one side, to the sensory retinal cells which perceive light and transmit visual information to the optic nerve. On the other side of the RPE is the choroid tissue, a vascularized region which supplies the overlying cells of the eye with water, nutrients and other compounds. The RPE plays many critical roles in maintaining vision including isolating the tissues of the eye from the general circulatory system, maintaining the proper ionic environment, processing discarded outer photoreceptor elements from the photoreceptor cells of the neural retina, and protecting the retina from excess light. Accordingly, the RPE is indispensable for vision by its maintenance of the photoreceptor cells which it supports.

Various retinal diseases may afflict this critical component of the eye. A primary pathology of the RPE is the dry form of age-related macular degeneration (AMD), a disease that gradually diminishes vision in the macula, the central region of the eye. AMD is a leading cause of vision loss in persons 60 years of age and older. It is estimated that in the United States 30% of people over age 75 suffer from some form of AMD. In the dry form of AMD, the accumulation of lipofuscin bisretinoids is observed. These species are vitamin A metabolites comprising undigested photoreceptor outer segment tips that are normally digested in the RPE. These lipofuscin bisretinoids, accumulate progressively as a byproduct of constant retinal chromophore recycling. Another symptom of dry AMD is the accumulation of lipid-protein aggregate called drusen, above and beneath the RPE, disrupting contact with the underlying nourishing choroid. The progressive accumulation of lipofuscin, bisretinoids, and drusen is associated with dysfunction and death of the RPE cells.

Other serious and blinding conditions of the retina associated with lipofuscin accumulation are known and afflict millions of subjects. Stargardt macular dystrophy, for example, including autosomal dominant Stargardt disease or autosomal recessive Stargardt disease, is a condition characterized by lipofuscin accumulation and macular degeneration. Other retinal conditions include, for example, neuronal ceroid lipofuscinosis, including Batten's Disease and Best vitelliform macular dystrophy.

Previous research elucidated the pathological cascade that underlies dry AMD and other conditions of the retina associated with lipofuscin accumulation, for example, as described in Toops et al., 2015. Cholesterol-mediated activation of acid sphingomyelinase disrupts autophagy in the retinal pigment epithelium. Mol. Biol. Cell 26: 1-14. These investigations determined that bisretinoids trap cholesterol and bis(monoacylglycero)phosphate, an acid sphingomyelinase (ASM) cofactor, within the RPE. It was further demonstrated that this promotes ASM activation, which in turn increases the accumulation of ceramide. Ceramide promotes microtubule acetylation, and this disrupts normal autophagosome traffic and impairs the vital autophagic flux in the RPE. It was further demonstrated that inhibition of ASM restores efficient autophagy and promotes the health of the RPE by disrupting the pathological cascade that is initiated by lipofuscin bisretinoids.

Therapeutic interventions based upon the foregoing discoveries are taught, for example, as described in United States Patent Application Publication Number US20150366876, Use of Inhibitors of Acid Sphingomyelinase to Treat Acquired and Inherited Retinal Degenerations, by Lakkaraju et al. Therein, it is demonstrated that the functional ASM inhibitor desipramine can effectively inhibit ceramide accumulation and the pathological cascade initiated thereby. Additionally, the therapeutic use of other ASM inhibiting agents was suggested, including the structural ASM inhibitor zoledronic acid, however demonstrations thereof were not provided.

Despite these recent and promising advances in the understanding and treatment of retinal conditions, clinical applications are not yet available and retinal conditions continue to afflict millions of persons worldwide. Accordingly, there remains a substantial need in the art for effective therapies in the treatment of retinal conditions such as dry AMD, Stargardt disease, and others.

As suggested by prior research, ASM inhibitors other than desipramine could potentially be useful in the treatment of retinal conditions. Zoledronic acid is a known ASM inhibitor and therefore could potentially be useful in the treatment of retinal conditions. However, to the knowledge of the inventors of the present disclosure, the evaluation of this agent and related bisphosphonate inhibitors of ASM in the context of retinal disease has not been previously performed. Zoledronic acid also inhibits farnesyl diphosphate synthase (FDPS), which is an enzyme responsible for cholesterol biosynthesis and protein prenylation.

Zoledronic acid, (1-Hydroxy-2-imidazol-1-yl-phosphonoethyl) phosphonic acid monohydrate, also called zoledronate, is a bisphosphonate, comprising

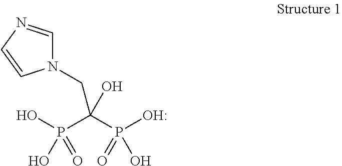

Among its various biological effects, zoledronic acid is an inhibitor of ASM (Roth, et al., Angew Chem Int Ed Engl. 48:7560-7563, 2009), as noted above, and also an inhibitor of farnesyl diphosphate synthase (FDPS).

Zoledronic acid also inhibits bone resorption by inhibiting osteoclastic activity and inducing osteoclast apoptosis. Zoledronic acid also binds to bone and blocks the osteoclastic resorption of mineralized bone. Clinically, zoledronic acid has been approved for treatment of osteoporosis and is sold in various forms such Aclasta™ (Novartis Pharmaceuticals) and Reclast™ (Novartis Pharmaceuticals). Zoledronic acid is also approved for use in treating Paget's disease.

In the context of cancer, zoledronic acid has also been approved for use in treating skeletal complications arising from certain cancers, for example, hypercalcemia of malignancy. Zoledronic acid can also induce apoptosis in cancer cells and is approved for treatment of multiple myeloma and bone metastases from certain solid tumors. Additionally, some types of solid tumors (breast cancer, prostate cancer, lung cancer) can also metastasize to the bone marrow. In the bone marrow, these cancers disrupt the function of the osteoclasts therein, promoting pathological bone resorption and inhibiting the formation of new bone. Zoledronic acid, by its inhibition of osteoclasts and bone resorption, as well as by inducing apoptosis in cancer cells, is approved to treat these pathologies, and is sold as Zometa™ (Novartis Pharmaceuticals).

Overall, zoledronic acid is considered a safe and well tolerated agent. However, despite the clear therapeutic utility of zoledronic acid, this agent has been associated with various negative side effects. Various sources note zoledronic acid side effects such as anemia, fatigue, muscle discomfort, and swelling of the lower extremities. Additionally, some subjects may be at risk of renal impairment from use of zoledronic acid, and its use may not be recommended for subjects with below-normal renal function such as CKD subjects. A rare but serious complication in certain subjects treated with bisphosphonates is osteonecrosis of the jaw, primarily in multiple myeloma subjects undergoing dental extractions. Additionally, the European Medicines Agency reported that atypical fractures may be a side effect of bisphosphonates. Furthermore, subjects with hypocalcemia may experience detrimental side effects from zoledronic acid.

As with many therapeutic agents, the administration of bisphosphonates also increases the risk of unfavorable interactions with other medications being administered to subjects. For example, increased risk of gastrointestinal bleeding results from an unfavorable interaction between zoledronic acid and drugs such as aspirin, celecoxib, and others. An increased risk of nephrotoxicity has been observed resulting from an unfavorable interaction between zoledronic acid and drugs such as acyclovir and cisplatin.

### BRIEF SUMMARY OF THE INVENTION

The inventions disclosed herein are based on the unexpected discovery that bisphosphonate ASM/FDPS inhibitors can be efficaciously administered to treat retinal diseases at ultra-low dosages not contemplated or suggested by prior uses of these agents. Specifically, the inventors of the present disclosure have determined that zoledronic acid and related compositions may be therapeutically effective at dosages which are at much lower, for example, as least 100 times lower, than standard doses thereof currently used in the clinic. Notably, the doses are about 2,000-fold lower than doses of desipramine previously demonstrated to achieve therapeutic effects in the retina.

The surprising and unexpectedly efficacy of these agents at ultra-low doses provides various benefits. In a first aspect, by the use of ultra-low doses, bisphosphonates such as zoledronic acid may be used to treat retinal conditions with a substantially reduced risk of the deleterious side effects or undesirable drug cross-interactions that have been observed when used at standard doses. By such risk reduction, the pool of subjects having retinal disease treatable by bisphosphonates is substantially expanded. These, and other therapeutic advantages are provided by the methods of the invention, as disclosed herein.

In one aspect, the disclosure features a method of treating a retinal disease in a subject in need of treatment therefor by administration to the subject of a therapeutically effective amount of a pharmaceutical composition comprising zoledronic acid or a derivative thereof, wherein the zoledronic acid or derivative is administered at an ultra-low dose. In some embodiments, the retinal disease is a condition mediated by lipofuscin accumulation in RPE cells. In some embodiments, the retinal disease is dry age-related macular degeneration or Stargardt macular dystrophy. In some instance, the retinal disease is neuronal ceroid lipofuscinosis, Batten's Disease, Bietti's crystalline dystrophy, Niemann-Pick disease Type C, Doyne's honeycomb dystrophy, Farber disease, or Best vitelliform macular dystrophy. In some embodiments, the pharmaceutical composition comprises or is incorporated within an implant; drug-eluting device, structure, or material; polymeric drug-eluting wafer; injectable hydrogel; or implantable hydrogel scaffold. In some embodiments, zoledronic acid or derivative is administered by intravitreal implant to deliver a dose of about 50 ng/day to about 50 μg/day to an eye. In some embodiments, zoledronic acid or derivative is administered to provide an intraocular concentration of zoledronic acid of at least 100 pM, 1 nM, 10 nM, 100 nM, 200 nM, 500 nM, or 1 μM. In some embodiments, zoledronic acid or the derivative thereof is administered as an eye drop solution or suspension, or ophthalmic ointment or gel at a dose of about 50 ng/day to about 50 μg/day to an eye. In some embodiments, zoledronic acid or the derivative is administered by suprachoroidal injection at about 50 ng/day to about 50 μg/day to an eye. In some embodiments, zoledronic acid or derivative is administered systemically to provide a dose between 0.001 and 2.0 mg, between 0.01 and 2.0 mg, or a dose between or 0.3 and 0.5 mg. In some embodiments, zoledronic acid or the derivative is administered systemically at a dose between 0.3 and 0.5 mg is administered at a dose selected from the group consisting of 0.001 mg, 0.02 mg, 0.03 mg, 0.04 mg, 0.05 mg, 0.06 mg, 0.07 mg, 0.08 mg, 0.09 mg, 0.10 mg, 0.20 mg, 0.30 mg, 0.40 mg, 0.50 mg, 0.60 mg, 0.70 mg, 0.80 mg, 0.90 mg, and 1.0 mg. In some embodiments, zoledronic acid or the derivative is administered systemically is administered at a dose between 100 ng to 10 μg per kg body mass, at a dose between 1.0 and 7.0 μg per kg body mass, or at a dose of about 5.0 μg per kg body mass. In some embodiments, zoledronic acid or the derivative is administered systemically in an amount of 0.1, 0.2, 0.3, 0.5, 1.0, 2.0, 3.0 4.0, 5.0, 6.0, 7.0, 8.0, 9.0, or 10.0 μg per kg body mass. In some embodiments, the administration is by a route comprising any of intravenous delivery, intramuscular delivery, intraperitoneal delivery, or subcutaneous delivery. In some embodiments, the pharmaceutical composition is administered at a frequency selected from the group consisting of: once per year, once per month, twice per month, weekly, twice weekly, every other day, daily, twice per day, and thrice per day. In some embodiments, the pharmaceutical composition comprises zoledronic acid and any of an excipient, carrier, diluent, release formulation, drug delivery or drug targeting vehicle, and additional active therapeutic agent. In some embodiments, the pharmaceutical composition comprises an adiponectin1 receptor agonist, for example adiporon. In some embodiments, zoledronic acid is co-administered with an adiponectin1 receptor agonist.

In a further aspect, the disclosure provides an intravitreal implant comprising zoledronic acid loaded with an amount of from 0.001 to 0.3 mg zoledronic acid. In some embodiments, the implant is loaded with 0.005 to 2.5 mg zoledronic acid.

In another aspect, the disclosure provides a topical ophthalmic preparation comprising 0.001-0.05 mg/dose of zoledronic acid. In some embodiments, the ophthalmic preparation of comprises 0.005-0.05 mg/dose of zoledronic acid.

In an additional aspect, the disclosure provides an injectable ophthalmic preparation comprising zoledronic acid at a concentration of 1 μg/ml-10 mg/ml. In some embodiments, the injectable ophthalmic preparation comprises zoledronic acid at a concentration of from 10 μg/ml-1 mg/ml. In some embodiments, the ophthalmic preparation comprises zoledronic acid conjugated to dendrimers or formulated as nano particles.

In a further aspect, the disclosure provides a method of treating a retinal disease in a subject in need of treatment therefor comprising administration to the subject a therapeutically effective amount of a pharmaceutical composition comprising adiporon to the subject; or a method of treating a retinal disease in a subject in need of treatment therefor by administration to the subject of a therapeutically effective amount of a pharmaceutical composition comprising a bisphosphonate ASM/FDPS inhibitor, wherein the bisphosphonate ASM/FDPS inhibitor is administered at an ultra-low dose. In some embodiments, the retinal disease is a condition mediated by lipofuscin accumulation in RPE cells. In some embodiments, the retinal disease is Stargardt macular dystrophy or dry age-related macular degeneration. In some embodiments, the retinal disease is neuronal ceroid lipofuscinosis, Batten's Disease, Bietti's crystalline dystrophy, Niemann-Pick disease Type C, Doyne's honeycomb dystrophy, Farber disease, or Best vitelliform macular dystrophy. In some embodiments, adiporon or the bisphosphonate ASM/FDPS inhibitor wherein is administered at a dose between 0.001 and 2.0 mg, at a dose between 0.1 and 1.0 mg, or at a dose between 0.3 and 0.5 mg. In some embodiments, adiporon or the bisphosphonate ASM inhibitor is administered at a dose selected from the group consisting of 0.001 mg, 0.002 mg, 0.003 mg, 0.004 mg, 0.005 mg, 0.006 mg, 0.007 mg, 0.008, mg, 0.009 mg, 0.01, 0.02 mg, 0.03 mg, 0.04 mg, 0.05 mg, 0.06 mg, 0.07 mg, 0.08 mg, 0.09 mg, 0.10 mg, 0.20 mg, 0.30 mg, 0.40 mg, 0.50 mg, 0.60 mg, 0.70 mg, 0.80 mg, 0.90 mg, and 1.0 mg. In some embodiments, adiporon or the bisphosphonate ASM/FDPS inhibitor is administered at a dose between 100 ng to 10 μg per kg body mass, at a dose of between 1.0 and 7.0 μg per kg body mass, or at a dose of about 5.0 μg per kg body mass. In some embodiments, adiporon or the bisphosphonate ASM/FDPS inhibitor is administered at a dose selected from the group consisting of 0.1, 0.2, 0.3, 0.5, 1.0, 2.0, 3.0 4.0, 5.0, 6.0, 7.0, 8.0, 9.0, and 10.0, μg per kg body mass. In some embodiments, the pharmaceutical composition is administered at a frequency selected from the group consisting of: once per year, once per month, twice per month, weekly, twice weekly, every other day, daily, twice per day, and thrice per day. In some embodiments, administration is by a route comprising any of: systemic delivery; local delivery; intravenous delivery; intramuscular delivery; intraperitoneal delivery; topical delivery; subcutaneous delivery; intraocular delivery; and topical delivery to the eye. In some embodiments, pharmaceutical composition comprises one or more bisphosphonate ASM/FDPS inhibitors or adiporon and any of an excipient, carrier, diluent, release formulation, drug delivery or drug targeting vehicle, and additional active therapeutic agent.

### BRIEF DESCRIPTION OF THE DRAWINGS

FIG. 1 depicts multiple pathways targeted by ZA to prevent retinal degeneration. ZA inhibits farnesyl diphosphate synthase (FDPS) and acid sphingomyelinase (ASM), which corrects multiple downstream pathological features of macular degenerations.

FIG. 2A-D provides data illustrating in vitro screening of ASM inhibitors in the RPE. 2A, Acetylated tubulin (green) staining in primary RPE cultures treated with the lipofuscin bisretinoid A2E (which activates ASM)±ASM inhibitors desipramine (des), amitriptyline (ami), fluoxetine (fluo) or zoledronic acid (ZA). All drugs used at 10 pM for 3 h, 48 h after A2E treatment. 2B, Quantification of acetylated tubulin fluorescence in A. *, significantly higher than in drug-treated or control cells, p<0.001. 2C, Stills from live imaging of autophagic flux (EGFP-mRFP-LC3) in cells treated with ZA. 2D, EGFP:mRFP ratios as a measure of autophagic flux in primary RPE. ***, p<0.001 compared to cells with A2E alone. Fex—fexofenadine; CBZ—cyclobenzaprine.

FIG. 3 provides data illustrating the effects of ZA treatment on ceramide accumulation in a mouse model of dry AMD. Top panels: RPE flatmounts from vehicle- or ZA-treated 18-month-old Abca4^{-/-} mice stained for ceramide. Lower panels: High-magnification images of boxes in flatmounts (both from the inferior portion of retina) and quantification of ceramide in the RPE. Actin (lighter lines of segments depicted in field) is stained using phalloidin. Mean±SEM, * p<0.05. n˜1500-1800 RPE from ≥3 mice per group.

FIG. 4 provides data illustrating the effects of ZA treatment on cholesterol accumulation in a mouse model of macular degeneration. Cholesterol in RPE cryosections from 14-month-old mice wildtype and Abca4^{-/-} labeled with filipin and quantification. Mean±SEM, * p<0.05; n˜1500-1800 RPE from ≥3 mice per genotype and treatment.

FIG. 5 provides data illustrating the effects of ZA treatment on complement-mediated mitochondrial fragmentation in the RPE in vitro. Representative 3D volume reconstructions from live imaging of Mitotracker-labeled mitochondrial networks in primary polarized RPE monolayers treated or not with A2E and exposed to normal human serum (NHS) as a source of complement. Cells were treated with 1 μM ZA for 3 hours after complement exposure and imaged immediately. Warmer colors—healthy mitochondria; cooler colors—fragmented mitochondria. Quantification of mitochondrial volumes. Mean±SEM, * p<0.05, one-way ANOVA and Tukey's multiple comparisons test. n=˜900 cells from 3 independent experiments.

FIG. 6 provides data illustrating the effects of ZA treatment on complement-mediated mitochondrial fragmentation in a mouse model of macular degeneration. Volume reconstructions and quantification of Tom20-labeled mitochondria in RPE flatmounts from 14-month-old mice (ZA 5 μg/kg i.p., three times/week for 8 weeks). Mean±SEM, * p<0.05. n˜1500-1800 RPE from ≥3 mice per genotype and treatment.

FIG. 7 provides data showing the effects of ZA treatment on subretinal microglia in a mouse mode of macular degeneration. Iba1-labeled subretinal microglia (gray) on mouse RPE flatmounts labeled with phalloidin and quantification of microglia per flatmount in 18-month-old Abca4^{-/-} mice treated with vehicle or ZA mice. Mean±SEM of subretinal microglia from three mice. *, p<0.05.

FIG. 8 provides data showing the effects of ZA treatment on photoreceptor degeneration in a mouse model of macular degeneration. Scotopic a-waves (which reflect photoreceptor integrity and function) and c-waves (which reflect RPE integrity and function) measured by electroretinograms (Diagnosys Celeris). Mean±SEM, * p<0.05. ≥3 mice per genotype and treatment

FIG. 9 provides illustrative data showing effects of adiporon treatment of RPE in vitro on complement-mediated mitochondrial fragmentation. Representative 3D volume reconstructions from live imaging of Mitotracker-labeled mitochondrial networks in primary polarized RPE monolayers treated or not with A2E and exposed to normal human serum (NHS) as a source of complement. Cells were treated with 1 μM Adiporon (ADP) for 3 hours after complement exposure and imaged immediately. Quantification of mitochondrial volumes. Mean±SEM, * p<0.05, one-way ANOVA and Tukey's multiple comparisons test. n=˜900 cells from 3 independent experiments.

### DETAILED DESCRIPTION

As used herein, “a”, “an”, and “the” include aspects with one member, but also include aspects with more than one member unless the context clearly dictates otherwise.

The terms “about” and “approximately” as used herein with respect to a given value generally mean a deviation from the stated value that is typically within 30% or within 20% of the stated value. For example, “about” with respect to doses or amounts is recognized by one of ordinary skill in the art to provide a pharmacological effect equivalent to that obtained from the specified dose or amount. In certain embodiments, the terms “about” and “approximately,” when used in this context, includes a dose or amount within 20%, within 15%, within 10%, or within 5%, of the specified dose or amount.

The present disclosure provides methods of treating a retinal disease in a subject by administration to the subject a pharmaceutically effective amount of a pharmaceutical composition comprising a bisphosphonate ASM/FDPS inhibitor, wherein the bisphosphonate ASM/FDPS inhibitor is administered at an ultra-low dose. As used herein, an ASM/FDPS inhibitor refers to a compound or agent that inhibits both acid sphingomyelinase (ASM) activity and farnesyl diphosphate synthase (FDPS) activity. In a related embodiment, the present disclosure further provides a pharmaceutical composition comprising a bisphosphonate ASM/FDPS inhibitor, for use in a method of treating a retinal disease in a subject, wherein the method comprises the administration of the bisphosphonate ASM/FDPS inhibitor at an ultra-low dose. In another aspect, the disclosure provides a method of making a medicament for the treatment of a retinal disease, comprising the use of a bisphosphonate ASM/FDPS inhibitor in an ultra-low dose.

Ultra-Low Doses. A primary aspect of the invention is the use of bisphosphonate inhibitors of ASM, e.g. zoledronic acid, at doses which are substantially lower than those currently used in the clinic. As used herein, a “dose” means an amount of a selected agent delivered at one time. For example, an example of a dose of sugar would be “4 grams” or “one 4-gram sugar cube.” As used herein, a dosage refers to a specific dose of a selected agent delivered over a selected period of time, optionally in a specified number of administrations, optionally at a specified frequency, optionally by a specified administration route. For example, an example of a dosage of sugar would be “eight grams of sugar per day, administered in two four-doses, one in the morning and one in the evening, administered orally.”

A “low dose” as used herein with respect to administration of an ASM/FDPS inhibitor, such as zoledronic acid, for treatment of a retinal disease comprises dramatically smaller, e.g., at least 5-fold lower, preferably 10-fold lower, or 100-fold lower doses of bisphosphonate ASM/FDPS inhibitor, e.g., zoledronic acid, than are commonly used in the clinic. In some embodiments, an ultra-low dose is at least 10-fold, preferably 100-fold lower than the doses of bisphosphonate ASM/FDPS inhibitor, e.g., zoledronic acid, that are commonly used in the clinic. For the treatment of osteoporosis, and for cancer related treatments, a standard dose of zoledronic acid is 4 or 5 mg, delivered at a dosage comprising a single dose of 4 or 5 mg zoledronic acid delivered intravenously, once per year. Such administration averages about 40-100 μg per kilogram body weight. As described in the Examples section herein, it was discovered by the inventors of the present disclosure that doses which are, in some embodiments, at least 100 times less than the established clinical dose can be used effectively to inhibit the lipofuscin-mediated pathologies in the RPE that underlie various retinal conditions.

In one implementation, the method of the invention encompasses the administration of a bisphosphonate ASM/FDPS inhibitor, e.g., zoledronic acid, to a human subject at a dose between 0.005 and 10 mg, or between 0.01 and 1 mg, for example, a dose between 0.5 mg and 1.0 mg, for example, a dose between 0.3 mg and 0.5 mg, for example, a dose in the range of 0.01-0.30 mg. Exemplary doses encompass, for example, 0.005 mg, 0.01 mg, 0.025 mg, 0.05 mg, 0.075 mg, 0.10 mg, 0.15 mg, 0.2 mg, 0.25 mg, and 0.3 mg. Equivalent doses may be calculated for smaller human subject or non-human animal subjects based on methodologies known in the art.

In one implementation, the method of the invention encompasses the administration of a bisphosphonate ASM/FDPS inhibitor, e.g., zoledronic acid, at a dose between 10 ng and 50 μg per kilogram body weight, for example, a dose between 100 ng and 20 μg per kilogram body weight, for example, a dose between 1.0 μg to 10 μg per kg body weight, for example, a dose in the range of 3-7 μg per kg body weight, or a dose of about 5 μg per kg body weight. Exemplary doses encompass, for example, 100 ng per kg body weight, 200 ng per kg body weight, 300 ng per kg body weight, 400 ng per kg body weight, 500 ng per kg body weight, 600 ng per kg body weight, 700 ng per kg body weight, 800 ng per kg body weight, 900 ng per kg body weight 1 μg per kg body weight, 2 μg per kg body weight, 3 μg per kg body weight, 4 μg per kg body weight, 5 μg per kg body weight and 6 μg per kg body weight, 7 μg per kg body weight, 8 μg per kg body weight, 9 μg per kg body weight, and 10 μg per kg body weight.

Typically, a dose range for a bisphosphonate ASM/FDPS inhibitor, e.g., zoledronic acid, for m² body surface area will be in the range of 0.001 mg/m² to 1.0 mg/m², for example in the range of 0.037 mg/m² to 0.37 mg/m², for example comprising any of about 0.05, 0.06, 0.07, 0.08, 0.09, 0.1.0, 0.2, 0.3, 0.4 or 0.5 mg/m². The human equivalent dose (HED) for zoledronic acid will range from 0.1 μg/kg to 1 μg/kg.

The doses disclosed above may be administered according to any number of suitable dosing regimens, dosages, dosage forms, and administration routes. Regarding frequency, the selected dose may be administered at any selected frequency, for example, once per year, once per month, once per week, once per day, etc. The course of treatment may be any, for example, one week, two weeks, three weeks, four weeks, two months, three months, six months, one year, or indefinitely as needed. Administration can also be performed on an as-needed basis determined by clinical assessment or adjusted in terms of amounts and/or treatment frequency based on clinical assessment. Dose amounts may additionally or alternatively be escalated from a lower dose to at least one higher dose over subsequent administrations. In some instances, dose amounts may additionally or alternatively be deescalated from a higher dose to a least one lower dose over subsequent administrations.

Additionally, administration may be by any selected administration route. In one implementation, the administration is systemic, for example comprising intravenous, intraperitoneal, subcutaneous, or oral administration. In such implementations, the administered bisphosphonate ASM/FDPS inhibitor, e.g., zoledronic acid, is typically administered at a relatively higher dose compared to local administration to the eye, in order to achieve therapeutically effective concentrations at the RPE, for example, in doses such as 0.01-1 mg for a human subject, doses of 1-30 μg per kg body weight, and doses of 0.037-1.0 mg per m² body surface area.

In another implementation, the administration comprises ocular administration, i.e. administration to a compartment of the eye. For example, the selected bisphosphonate ASM/FDPS, inhibitor, e.g., zoledronic acid, may be administered by intraocular delivery, for example, by methods such as suprachoroidal injection, subconjunctival injection, intravitreal injection, sub-retinal, or sub-tenon injection. In such implementations, the administered bisphosphonate ASM/FDPS inhibitor will bypass the blood brain barrier (BBB) and systemic dilution and clearance, and may achieve therapeutically effective concentrations at the RPE with relatively lower doses than would be administered systemically. For example, in some embodiments, doses delivered by intraocular delivery may comprise 0.001-0.1 mg for a human subject, doses of 0.05-2 μg per kg body weight, and doses of 0.001-0.1 mg per m² body surface area.

In a related implementation, the method of treatment using intraocular administration may further include an anti-inflammatory regimen in the peri-injection period. The anti-inflammatory regimen may include steroids and non-steroidal anti-inflammatories alone or in combination. The anti-inflammatory medications may be administered via an oral, intraocular and/or ocular topical route alone or in combination.

In a related implementation, the administration to the eye is topical administration to the eye, for example by the use of gels, ophthalmic ointments, or drops applied to the outer surface of the eye. Exemplary dosage forms include eyedrops or gels, for example, comprising solutions, suspensions, emulsions, or other preparations of the selected bisphosphonate ASM/FDPS inhibitor, e.g., zoledronic acid, in a carrier. In such implementations, it may be expected that the delivery will be more efficient than for systemic delivery but less efficient than by intraocular delivery, as the agent must traverse the vitreous membrane and the ocular fluid and photoreceptors to reach the RPE. In some embodiments, topical doses applied to the eye may encompass 0.01-0.5 mg for a human subject, doses of 0.5-8 μg per kg body weight, and doses of 0.0185-0.3 mg per m² body surface area.

In some embodiments, doses for humans are based on animal doses, e.g., based on dose-scaling conversions that are frequently used in the art. (see, for example Nair & Jacob, J. Basic and Clin. Pharmac 2016; 7:27-31). Thus, for example, to convert an animal dose in mg/kg to a human equivalent dose in mg/kg, an animal dose may be divided by 12.3.

In some embodiments, zoledronic acid is formulated as an ophthalmic solution or suspension for administration as eye drops, e.g., as a solution, suspension, liposomal formulation, mucoadhesive polymer, micelle, nanoparticle and the like (See, e.g., Kim et al, Drug Delivery and Translational Research 12:826-837, 2022). In some embodiments the ophthalmic solution or suspension provides a dose of zoledronic acid per eye of about 10 ng/day to about 250 μg/day. In some embodiments the ophthalmic solution or suspension provides a dose of zoledronic acid per eye of about 10 ng/day to about 100 μg/day. In some embodiments, eye drops are formulated to provide a dose of zoledronic acid per eye of about 50 ng/day to about 50 μg/day. In some embodiments, eye drops are formulated to provide a dose of zoledronic acid per day of about 100 ng/day to about 50 μg/day. In some embodiments, eye drops are formulated to provide a dose of zoledronic acid per eye of about 50 ng/day, or 100 ng/day, to about 10 μg/day. In some embodiments, eye drops are formulated to provide a dose of zoledronic acid per eye of at least about 50 ng/day, at least 100 ng/day, at least 200 ng/day, at least 250 ng/day, at least 300 ng/day, at least 350 ng/day, at least 400 ng/day, at least 500 ng/day, at least 600 ng/day, at least 700 ng/day, at least 800 ng/day, at least 900 ng/day, at least pg/day, at least 1.5 μg/day, at least 2 μg/day, at least 2.5 μg/day, at least 3 μg/day, at least 4 μg/day, at least 5 μg/day, at least 10 μg/day, at least 15 μg/day, at least 20 μg/day, or at least 25 μg/day, but no more than about 50 μg/day. In some embodiments, eye drops are formulated to provide a consistent intraocular concentration of zoledronic acid of at least 100 pM, 1 nM, 10 nM, 100 nM, 200 nM, 500 nM, or 1 μM. In some embodiments, an ophthalmic solution is administered once per day to provide the indicated dose per day. In some embodiments, the ophthalmic solution is administered more than once a day, e.g., twice, or three times a day to provide the indicated dose day. In some embodiments, an ophthalmic solution can be administered at least daily, two times a week, three times a week, four times a week, or more. In some embodiments, zoledronic acid can be administered once a week or once every two weeks. In some embodiments, zoledronic acid can be administered once a month or twice a month.

In some embodiments, zoledronic acid is delivered to the eye using an intravitreal implant. In such embodiments, zoledronic acid can be incorporated into an implant to provide release of zoledronic acid over a period of time for up to years, e.g., up to 2 year or 3 years. In some embodiments, an implant can be loaded with an amount from about 0.001 to about 0.3 mg of zoledronic acid. In some embodiments, zoledronic acid can be incorporated into an implant to provide release of zoledronic acid over a period of less than of time from weeks to months, for example, 2-4 weeks, or 4-6 weeks, or 6-8 weeks, or 3-6 months, or 6-12 months or 12-18 months. In some embodiments an implant may be a biodegradable polymer. In some embodiments, an implant is loaded with an amount of zoledronic acid, to provide release at a dose of about 0.001 μg/day to about 2.5 μg/day, or release of a dose of about 0.005 μg/day to about 2.0 μg/day. In some embodiments, an implant is loaded with an amount of zoledronic acid to provide a consistent intraocular concentration of zoledronic acid of at least 100 pM, 1 nM, 10 nM, 100 nM, 200 nM, 500 nM, or 1 μM. In some embodiments, an implant is loaded with zoledronic acid, to provide a dose of about of about 10 ng/day to about 250 μg/day. In some embodiments, an implant is loaded to provide a dose of zoledronic acid of about 50 ng/day to about 100 μg/day. In some embodiments, an implant is loaded to provide a dose of zoledronic acid of about 100 ng/day to about 50 μg/day. In some embodiments, an implant is loaded to provide a dose of zoledronic acid of about 100 ng/day to about 10 μg/day. In some embodiments, an implant is loaded to provide a dose of zoledronic acid of at least about 50 ng/day, at least 100 ng/day, at least 200 ng/day, at least 250 ng/day, at least 300 ng/day, at least 350 ng/day, at least 400 ng/day, at least 500 ng/day, at least 600 ng/day, at least 700 ng/day, at least 800 ng/day, at least 900 ng/day, at least 1 μg/day, at least 1.5 μg/day, at least 2 μg/day, at least 2.5 μg/day, at least 3 μg/day, at least 4 μg/day, at least 5 μg/day, at least 10 μg/day, at least 15 μg/day, at least 20 μg/day, or at least 25 μg/day, but no more than about 50 μg/day or 100 μg/day. In some embodiments, the sustained release implant is an immiscible or partially immiscible liquid, including but not limited to benzyl benzoate and/or silicone oil. Such an embodiment may contain a suspension, emulsion or solution of zoledronic acid

In some embodiments, suprachoroidal injections are used to administer zoledronic acid. In some embodiments, a zoledronic acid pharmaceutical composition is employed in which zoledronic acid is conjugated to dendrimers (see, .e.g., Pitha et al, Biomacromolecules 24:1355-1365, 2023) or is provided as a nanoparticle formulation (see, e.g., Pitha et al, Biomacromolecules 24:1355-1365, 2023; Laradji et al, Polymers 13(19), 3324, 2021). In some embodiments, an injectable solution or suspension is formulated to provide zoledronic acid at a concentration of 1 μg/ml-10 mg/ml and delivered by suprachoroidal injection, or intravitreal injection. In some embodiments, an injectable solution or suspension comprising zoledronic acid, e.g., conjugated to dendrimers or in a nanoparticle formulation, is formulated to deliver zoledronic acid in an amount of about 0.1-1 mg/eye. In some embodiments, the amount of zoledronic acid delivered by injection to an eye, e.g., suprachoroidal or intravitreal injection, is a dose of about of about 10 ng/day to about 50 μg/day. In some embodiments, the amount of zoledronic acid delivered by injection to an eye, e.g., suprachoroidal or intravitreal injection, is a dose of about of about 10 ng/day to about 50 μg/day. In some embodiments, the dose of zoledronic acid for injection is about 100 ng/day to about 50 ug/day. In some embodiments, dose of zoledronic acid for injection is about 100 ng/day to about 10 μg/day. In some embodiments, an injectable is loaded to provide a dose of zoledronic acid of at least about 50 ng/day, at least 100 ng/day, at least 200 ng/day, at least 250 ng/day, at least 300 ng/day, at least 350 ng/day, at least 400 ng/day, at least 500 ng/day, at least 600 ng/day, at least 700 ng/day, at least 800 ng/day, at least 900 ng/day, at least 1 μg/day, at least 1.5 μg/day, at least 2 μg/day, at least 2.5 μg/day, at least 3 μg/day, at least 4 μg/day, at least 5 μg/day, at least 10 μg/day, at least 15 μg/day, at least 20 μg/day, or at least 25 μg/day and less than 100 μg/day, preferably about 50 μg/day of less per day. In some embodiments, an injectable composition, e.g., for suprachoroidal or intravitreal injection, is formulated to provide a consistent intraocular concentration of zoledronic acid of at least 100 pM, 1 nM, 10 nM, 100 nM, 200 nM, 500 nM, or 1 μM.

In some embodiments, an ophthalmic or systemic composition may be administered using a pre-filled syringe.

Bisphosphonate ASM/FDPS inhibitors. The methods of the invention encompass the use of bisphosphonate ASM/FDPS inhibitors to treat a selected retinal condition. In a primary embodiment, the bisphosphonate ASM/FDPS inhibitor is zoledronic acid or pharmaceutically effective variant thereof. Zoledronic acid, as used herein, encompasses any therapeutically active form of zoledronic acid, including Structure 1, pharmaceutically effective salts thereof, anhydrous, hygroscopic, and hydrous forms thereof, lipophilic derivatives thereof and chemically related derivatives thereof.

In one embodiment, the zoledronic acid is provided as a salt, for example, any of arginine salts, calcium salts, chromium salts, citrulline salts, cobalt salts, copper salts, creatine salts, glutamine salts, histidine salts, iron salts, isoleucine salts, leucine salts, lithium salts, lysine salts, magnesium salts, manganese salts, molybdenum salts, ornithine salts, potassium salts, selenium salts, sodium salts, zinc salts, and combinations of the foregoing.

In one embodiment, the bisphosphonate ASM/FDPS inhibitor comprises a derivative of zoledronic acid as disclosed in U.S. Pat. No. 4,939,130, “Substituted alkanediphosphonic acids and pharmaceutical use,” by Jaeggi and Wilder. In one embodiment, the bisphosphonate ASM/FDPS inhibitor comprises a derivative of zoledronic acid as disclosed in PCT International Patent Application Publication Number WO/2012071517, “Novel Crystalline Forms,” by Hanna et al. In one embodiment, the bisphosphonate ASM inhibitor comprises a derivative of zoledronic acid as disclosed in United States Patent Application Publication Number US20100056481, “Crystalline forms of zoledronic acid,” by Glausch et al. In another embodiment, the bisphosphonate ASM/FDPS inhibitor comprises a derivative of zoledronic acid conjugated to lysine-linked deoxycholic acid to increase oral absorption as described by Jeon et al., 2016. In another embodiment, the bisphosphonate ASM/FDPS inhibitor comprises a composition as described in U.S. Pat. No. 9,682,091, entitled “Oral Forms of a Phosphonic Acid Derivative.”

In one implementation, the bisphosphonate ASM/FDPS inhibitor comprises a form of zoledronic acid configured for oral delivery. Oral delivery conveniently bypasses the inconvenience and morbidity risk of ocular injection. For example, in one embodiment, the bisphosphonate ASM/FDPS inhibitor comprises an orally bioavailable form of zoledronic acid as disclosed in: United States Patent Application Publication Number US20140051669, “Compositions of Zoledronic Acid or Related Compounds for Treating Disease” by Tabuteau et al.; or PCT International Patent Application Publication Number WO2013015599, “Pharmaceutical Composition for Oral Administration Comprising Bisphosphonic Acid or its Salt,” by Kim et al.

Adiporon In a further aspect of the present disclosure, adiporon (PubChem CID 16307093) is used to treat a selected retinal condition. Adiporon (Case number is a selective, orally active, synthetic small-molecule agonist of the adiponectin receptor 1 (AdipoR1) and adiponectin receptor 2 (AdipoR2) synthetic adiponectin-receptor agonist. In some embodiments, adiporon is administered in an amount described herein for administration of bisphosphonate ASM/FDPS inhibitor. In some embodiments, Adiporon is employed at a dose of 10-1000-fold less than that used for other indications.

Treatment of Retinal Conditions. The methods of the invention are directed to the treatment of a retinal condition in a subject in need or treatment therefor by administration of a low dose of a bisphosphonate ASM/FDPS inhibitor.

Regarding subjects, the subject may be any animal subject in need of treatment for a retinal condition. The subject may be any animal species. In a primary embodiment, the subject is a human, for example, a human patient. In other implementations, the subject is a non-human animal, for example, a veterinary subject, pet, livestock, or test animal. Exemplary non-human animals include mice, rats, pigs, horses, cows, dogs, cats, non-human primates and others.

The subject may be a subject suffering from or diagnosed with a selected retinal condition. The subject may be a subject suspected of having a selected retinal condition. In one embodiment, the subject is a subject at risk of developing a selected retinal condition. In one embodiment, the subject is an aged subject, for example, a human subject of at least 50 years, at least 55 years, at least 60 years, or at least 65 years of age. In one embodiment, the subject is a subject having one or more genetic markers indicative of risk of a retinal condition, or is a subject with a family history of a retinal condition at risk of inheritance thereof. In one implementation, the subject is at risk, suspected of having, or suffering from dry age-related macular degeneration.

In some instance, the subject is a human child, teenager, or young adult, e.g., 25 years of age or younger at risk for, suspected of having, or diagnosed with a retinal disease. Embodiments, the subject is at risk, suspected of having, or suffering from Stargardt macular dystrophy, also referred to herein as Stargardt disease. In some embodiments, the subject is an adult above the age of 25 that is at risk for Stargardt macular dystrophy or is suspected of having, or has, Stargardt macular dystrophy. In some embodiments, the subject has autosomal dominant Stargardt disease. In some instances, the subject has autosomal recessive Stargardt disease.

Regarding the retinal conditions treatable by the methods of the invention, the retinal condition may be any condition of the retina wherein RPE dysfunction, microglial activation, or photoreceptor deficits are known or suspected, including conditions characterized or involving any of lipofuscin accumulation in RPE cells, cholesterol accumulation in RPE cells, aberrant activation of ASM or FDPS in RPE cells, aberrant ceramide production therein, aberrant Rab GTPase prenylation in the RPE, aberrant acetylation of microtubules in RPE cells, impaired autophagy in RPE cells drusen accumulations or like deposits above or beneath the RPE, complement mediated injury of mitochondria in the RPE, presence of subretinal microglia, or loss of photoreceptors or functional deficits in photoreceptors. As noted, in a primary implementation, the condition is Dry AMD. In other implementations, the retinal condition is Stargardt macular dystrophy, for example, including autosomal dominant or autosomal recessive Stargardts disease; Doyne's honeycomb dystrophy; an acid ceramidase disease such as Farber disease; a disease of cholesterol and ceramide accumulation and autophagy defects, such as Niemann Pick Type C disease; and disease such as Batten's Diseases (neuronal ceroid lipofuscinoses), Best vitelliform macular dystrophy; retinitis pigmentosa; and Bietti's crystalline dystrophy.

The methods of the invention encompass the treatment of a selected retinal condition. As used herein, “treatment” will encompass achieving any number of therapeutic effects and outcomes with respect to the selected retinal condition, including, for example: ameliorating symptoms associated of the selected retinal condition; slowing the progression of the selected retinal condition; preventing further damage to the RPE by the selected retinal condition; improving RPE function; maintaining, improving or restoring photoreceptor health; maintaining, improving or restoring vision; or any other reduction in morbidity associated with the selected retinal condition. Treatment, as used herein, will further encompass prevention of the selected retinal condition. For example, a preventative treatment may encompass achievement of any of: preventing or delaying the onset of the selected retinal condition in an at-risk subject; maintaining normal vision or function of the RPE; or otherwise preventing onset of the selected retinal condition. Treatment, as used herein, further encompass prevention of progression to geographic atrophy, slowing progression of geographic atrophy, treatment of neovascular prevention of progression to neovascular AMD, reduction of number and/or frequency of other treatments for AMD (such as anti-VEGF injections, complement inhibitor(s), etc.), change in total geographic atrophy (GA) area based on fundus autofluorescence, reduction in photoreceptor loss (e.g. as measured by ellipsoid zone attenuation area), change in reading speed under standard and low light, change in contrast sensitivity, change in retinal sensitivity on perimetry, change in patient-reported outcomes, change in dark adaptation, change in drusen volume, changes in pigment mottling, frank macular atrophy, bull's eye maculopathy, or fundus flecks, changes in electroretinogram (ERG), and/or structural changes as measured by optical coherence tomography (OCT).

Treatment further encompasses any inhibition of pathological processes underlying the selected retinal condition. Exemplary treatment effects include, for example, reducing lipofuscin accumulation in the RPE, reducing cholesterol accumulation in RPE cells, decreasing ASM and/or FDPS activity in RPE cells, reducing aberrant ceramide production therein, reducing aberrant Rab prenylation, reducing aberrant acetylation of microtubules in RPE cells, improving or restoring autophagic capacity, activity, and flux in RPE cells, reducing the formation of drusen accumulations or like deposits, reducing complement-mediated injury of mitochondria in the RPE, preventing or rescuing microglial migration into the subretinal space, or preventing or rescuing photoreceptor loss and functional deficits. As demonstrated in Example 3 herein, bisphosphonate ASM and FDPS inhibition by zoledronic acid advantageously reduces lipofuscin, cholesterol, and ceramide accumulation, in contrast to previously explored agents such as desipramine, which decreases ceramide alone. Accordingly, in one embodiment the scope of the invention further encompasses a method of reducing lipofuscin accumulation, cholesterol accumulation, and/or ceramide accumulation by the administration of zoledronic acid or a derivative thereof, for example, at an ultra-low dose.

Treatments of the invention encompass the administration of one or more bisphosphonate ASM/FDPS inhibitors to a subject in a therapeutically effective amount. In one measure, a therapeutically effective amount is an amount of a bisphosphonate ASM/FDPS inhibitors that is sufficient to induce a measurable therapeutic effect. The therapeutic effect may be the attainment of a selected physiological outcome or state, for example, decreased lipofuscin, cholesterol, or ceramide accumulation; decreased ASM and FDP activity; improvement of autophagic flux in the RPE; reduced drusen formation by RPE cells; reduced complement-mediated injury of mitochondria in RPE cells; improved health or function of the RPE; improved health or function of photoreceptors; decreased activation of microglia; and improved vision. As used herein, a “subject” refers to any mammal, including non-human primates, mice, rats, rabbits, pigs, horses, cows, goats, sheep, dogs, cats and the like. In preferred embodiments, the subject is a human.

Pharmaceutical Compositions. The methods and compositions of the invention encompass the administration of one or more bisphosphonate ASM/FDPS inhibitors, e.g., zoledronic acid. The one or more bisphosphonate ASM/FDPS inhibitors may be formulated in what will be termed “pharmaceutical compositions.” As used herein, the pharmaceutical composition will comprise one or more bisphosphonate ASM inhibitors and may further comprise any number of additional compositions of matter, including excipients, carriers, diluents, release formulations, drug delivery or drug targeting vehicles, as well as additional active therapeutic agents. The pharmaceutical compositions of the invention may be formulated to be compatible with the selected route of administration, for example, oral, injected, or ocular delivery.

The pharmaceutical compositions of the invention may comprise one or more bisphosphonate ASM/FDPS inhibitors, e.g., zoledronic acid, in combination with drug delivery compositions. Drug delivery compositions encompass any moieties, materials, or other compositions of matter that facilitate the delivery of the ASM/FDPS inhibitor, e.g., zoledronic acid, to the RPE. In some implementations, the drug delivery composition facilitates targeting of the ASM/FDPS inhibitor, e.g., zoledreonic acid, to the CNS, PNS, or other selected target compartment of the nervous system. In some implementations, the delivery composition comprises a composition of matter that facilitates crossing of the blood brain barrier. In some implementations, the delivery compositions comprise a composition of matter that facilitates transport across the blood-retinal barrier (BRB).

The pharmaceutical compositions may encompass any form of combination, including functionalization of the bisphosphonate ASM/FDPS inhibitor, e.g., zoledronic acid, with the delivery composition, conjugation of the bisphosphonate ASM/FDPS inhibitor to the delivery composition; admixture of the bisphosphonate ASM/FDPS inhibitor with the delivery composition; encapsulation or infusion of the bisphosphonate ASM/FDPS inhibitor within the delivery composition, or any other combination.

In one implementation, the delivery compositions comprise materials that facilitate the bisphosphonate ASM/FDPS inhibitor, e.g., zoledronic acid, crossing of the BBB or BRB. For example, the delivery composition may comprise ligands that facilitated transcytosis across the BBB through brain endothelial cells to the basolateral side, such as anti-transferrin receptor antibodies or antigen-binding fragments thereof, for example OX26 antibodies, Angiopep2 or like polypeptides, ApoE proteins and mimetics thereof, diphtheria toxin, and surfactants. Additional targeting moieties include BBB-crossing peptides, such as those described in Van Dorpe et al., Brainpeps: The blood-brain barrier peptide database, Brain Structure and Function, 2012, 217(3), 687-718. Additionally, the delivery composition may comprise a hypertonic or hyperosmolar formulation to facilitate BBB or BRB transport.

In one implementation, the delivery compositions comprise carriers to which the bisphosphonate ASM/FDPS inhibitor, e.g., zoledronic acid, is conjugated, encapsulated within, or otherwise combined with to facilitate crossing of the BBB and/or delivery to target RPE cells. Exemplary carriers include: liposomes; extracellular vesicles or synthetic mimetics thereof, such as exosomes; red blood cells modified with a bisphosphonate ASM/FDPS inhibitor; microspheres, such as poly(lactic-co-glycolic acid) (PLGA) microspheres; and other drug delivery nanoparticles such as PLGA-PEG nanoparticles, alginate or chitosan nanoparticles, silica nanoparticles, and iron oxide nanoparticles. The carrier molecules or compositions may further be functionalized with ligands that promote crossing of the BBB or BRB, such as anti-tfR antibodies, Angiopep2 or like polypeptides, ApoE proteins and mimetics thereof; diphtheria toxin, and surfactants, as described above.

In some embodiments, a bisphosphophonate ASM/FDPS inhibitor, e.g., zoledronic acid, is administered as a drug-antibody conjugate, for example, comprising an antibody or antigen-binding fragment thereof targeted to a ligand present on the RPE.

In one embodiment the delivery composition comprises or is incorporated within an implant, for example, a drug-eluting implant placed within the target tissue, for example, the eye. Exemplary implants include, for example, implants made of a biodegradable material, such as PLGA, polymeric drug-eluting wafers, polymeric drug-eluting rods, injectable hydrogels, implantable hydrogel scaffolds, hydrophilic microsphere-based systems, cyclodextrin-based systems, polymeric micelle-based systems, and other drug-eluting implants known in the art. Exemplary implants for delivery of agents to the RPE include those described in: WO2012177968, “A scaffold for subretinal cell transplantation and drug delivery,” by Tao et al. Illustrative polymers and implants for ocular drug delivery are additionally described by Allyn et al, Frontiers in Medicine, 8: Article 787644, January 2022; Cao et al, Drug Discovery Today 24(8), 1694-1700 2019).

In one implementation, a delivery composition comprises an intravitreal implant comprising zoledronic acid loaded with an amount of zoledronic acid ranging from 0.001-0.3 mg. In some embodiments, the implant is loaded with 0.005 to 2.5 mg zoledronic acid.

In a further embodiment, a delivery composition comprises a topical ophthalmic preparation comprising 0.001-0.05 mg/dose of zoledronic acid. In some embodiments, the preparation comprises 0.005-0.05 mg/dose of zoledronic acid. In some embodiments, the topical ophthalmic preparation is a solution or suspension applied as eye drops.

In a further embodiment, a delivery composition comprises an injectable ophthalmic preparation, e.g., suitable for suprachoroidal or intravitreal injection comprising zoledronic acid at a concentration of 1 μg/ml-10 mg/ml. In some embodiments, the injectable ophthalmic preparation comprises zoledronic acid at a concentration of from 10 μg/ml-1 mg/ml. In some embodiments, the injectable ophthalmic preparation comprises zoledronic acid conjugated to dendrimers or formulated as nano particles.

The methods of the invention will be understood to further encompass the combined administration of a bisphosphonate ASM/FDPS inhibitor, e.g., zoledronic acid, and one or more additional active agents for treatment of a retinal condition. Additional treatments, e.g., for the treatment of macular degeneration or Starsgardt disease, include but are not limited to, anti-VEGF treatments (including but not limited to rolucizumab, aflibercept, ranibizumab, pegaptanib sodium, faricimab-svoa and bevacizumab), complement inhibitors (including but not limited to pegcetacoplan and avacincaptad pegol), MCO 010, deuterated retinol, emixustat, MA09 hRPE, STG-001, tinlarebant, QR1011 and REV 0100. Combined administration, as used herein may encompass any combination of bisphosphonate ASM/FDPS inhibitor administration and the one or more additional treatments of the retinal condition. For example, the timing of the administration of ASM/FDPS inhibition treatment and one or more additional treatments may be determined by one of skill in the art. In various implementations, the ASM/FDPS inhibition treatment and additional treatment(s) are administered any of contemporaneously, sequentially, or alternating. In one embodiment, the first and second treatments are applied contemporaneously, i.e. simultaneously or overlapping in time. In one embodiment, the first and second treatments are administered in a pharmaceutical composition comprising a combination product, comprising a bisphosphonate ASM/FDPS inhibitor and an additional agent, for example, administered in a single dosage form. In one embodiment, the one or more additional treatments comprises the administration of an adiponectin1 receptor agonist, for example, as described in Kim et al, 2022. Adiponectin receptor agonist ameliorates cardiac lipotoxicity via enhancing ceramide metabolism in type 2 diabetic mice. CellDeath & Disease 13: 282. In one embodiment, the adiponectin1 receptor agonist is adiporon. In one embodiment, the pharmaceutical compositions of the invention comprises a combination products comprising a bisphosphonate ASM/FDPS inhibitor in combination with one or more adiponectin1 receptor agonists, for example, adiporon.

The pharmaceutical compositions of the invention may be formulated in any number of dosage forms. Exemplary dosage forms include: liquid solutions; sachets, capsules, or tablets, each containing a predetermined amount of the active ingredient, as solids or granules; suspensions in a liquid; emulsions; aqueous and non-aqueous solutions; isotonic sterile injection solutions; compositions stored in a freeze-dried, lyophilized condition; and other dosage forms known in the art. In one embodiment the dosage form is formulated for topical application to the eye, including eye drops, ophthalmic ointments or gels.

Illustrative embodiments include, but are not limited to, the following:

Embodiment 1: A method of treating a retinal disease in a subject in need of treatment therefor by administration to the subject of a pharmaceutically effective amount of a pharmaceutical composition comprising a bisphosphonate ASM/FDPS inhibitor, wherein the bisphosphonate ASM/FDPS inhibitor is administered at an ultra-low dose.
Embodiment 2: The method of Embodiment 1, wherein the retinal disease is a condition mediated by lipofuscin accumulation in RPE cells.
Embodiment 3. The method of Embodiment 1, wherein the retinal disease is dry age-related macular degeneration.
Embodiment 4. The method of Embodiment 1, wherein the retinal disease is selected from the group consisting of Stargardt macular dystrophy, neuronal ceroid lipofuscinosis, Batten's Disease, Bietti's crystalline dystrophy, Niemann-Pick disease Type C, Doyne's honeycomb dystrophy, Farber disease, and Best vitelliform macular dystrophy.
Embodiment 5. The method of Embodiment 1, wherein the bisphosphonate ASM/FDPS inhibitor is zoledronic acid or a derivative thereof.
Embodiment 6. The method of Embodiment 1, wherein the bisphosphonate ASM/FDPS inhibitor is administered at a dose between 0.005 and 2.0 mg.
Embodiment 7. The method of Embodiment 6, wherein the bisphosphonate ASM/FDPS inhibitor is administered at a dose between 0.1 and 1.0 mg.
Embodiment 8. The method of Embodiment 7, wherein the bisphosphonate ASM/FDPS inhibitor is administered at a dose between 0.3 and 0.5 mg.
Embodiment 9. The method of Embodiment 6, wherein the bisphosphonate ASM/FDPS inhibitor is administered at a dose selected from the group consisting of 0.005 mg, 0.006 mg, 0.007 mg, 0.008, mg, 0.009 mg, 0.01 mg, 0.02 mg, 0.03 mg, 0.04 mg, 0.05 mg, 0.06 mg, 0.07 mg, 0.08 mg, 0.09 mg, 0.10 mg, 0.20 mg, 0.30 mg, 0.40 mg, 0.50 mg, 0.60 mg, 0.70 mg, 0.80 mg, 0.90 mg, and 1.0 mg.
Embodiment 10. The method of Embodiment 1, wherein the bisphosphonate ASM/FDPS inhibitor is administered at a dose between 100 ng to 10 μg per kg body mass.
Embodiment 11. The method of Embodiment 10, wherein the bisphosphonate ASM/FDPS inhibitor is administered at a dose between 1.0 and 7.0 μg per kg body mass.
Embodiment 12. The method of Embodiment 11, wherein the bisphosphonate ASM/FDPS inhibitor is administered at a dose of about 5.0 μg per kg body mass.
Embodiment 13. The method of Embodiment 10, wherein the bisphosphonate ASM/FDPS inhibitor is administered at a dose selected from the group consisting of 0.1, 0.2, 0.3, 0.5, 1.0, 2.0, 3.0 4.0, 5.0, 6.0, 7.0, 8.0, 9.0, and 10.0, μg per kg body mass.
Embodiment 14. The method of any of Embodiments 1-13, wherein the pharmaceutical composition is administered at a frequency selected from the group consisting of: once per year, once per month, twice per month, weekly, twice weekly, every other day, daily, twice per day, and thrice per day.
Embodiment 15. The method of any of Embodiments 1-4, wherein the administration is by a route comprising any of: systemic delivery; local delivery; intravenous delivery; intramuscular delivery; intraperitoneal delivery; topical delivery; subcutaneous delivery; intraocular delivery; suprachoroidal delivery, and topical delivery to the eye.
Embodiment 16. The method of any of Embodiments 1-15, wherein the pharmaceutical composition comprises one or more bisphosphonate ASM/FDPS inhibitors and any of an excipient, carrier, diluent, release formulation, drug delivery or drug targeting vehicle, and additional active therapeutic agent.
Embodiment 17. The method of Embodiment 16, wherein the pharmaceutical composition comprises an adiponectin1 receptor agonist.
Embodiment 18. The method of Embodiment 17, wherein the adiponectin1 receptor agonist comprises adiporon.
Embodiment 19. The method of any of Embodiments 1-18, wherein the pharmaceutical composition comprises any of a solution; suspension in a liquid; emulsion; isotonic sterile injection solutions; compositions in lyophilized condition; eye drop solution; or ophthalmic ointment or gel.
Embodiment 20. The method of any of Embodiments 1-19, wherein the pharmaceutical composition comprises or is incorporated within an implant; drug-eluting device, structure, or material; polymeric drug-eluting wafer; injectable hydrogel; and implantable hydrogel scaffold.
Embodiment 21. The method of Embodiment 1, wherein the bisphosphonate ASM is co-administered with an adiponectin1 receptor agonist.
Embodiment 22. The method of Embodiment 21, wherein the adiponectin1 receptor agonist is adiporon.

### EXAMPLES

### Example 1. In Vitro Screening of ASM Inhibitors

An investigation for the evaluation of ASM inhibitors for the treatment of retinal conditions associated with pathological lipofuscin accumulation was initiated.

Primary RPE cell cultures were established with cells from porcine eyes. The cultured RPE cells were treated with the lipofuscin bisretinoid A2E, previously shown to activate ASM. Cultures were subsequently treated with various ASM inhibitors, and measures of lipofuscin-mediated injury were assessed. The ASM inhibitors were desipramine, amitriptyline, fluoxetine, and zoledronic acid, each applied at 10 μM, added to the cultures 48 hours after A2E treatment.

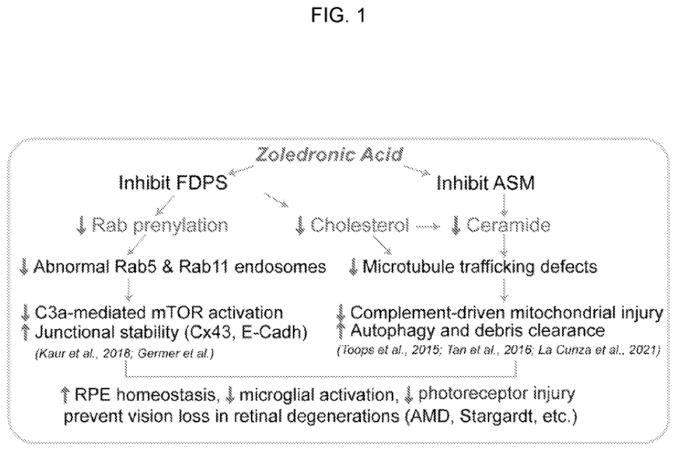

Acetylated tubulin in the cultured RPE cells was assessed by immunostaining with a specific antibody. As previously described, for example, in FIG. 1 and in Toops et al., 2015, lipofuscin accumulation traps cholesterol within RPE cells, triggering aberrant activation of ASM. ASM activity results in the accumulation of ceramide, which in turn promotes acetylation of microtubules. This acetylation of microtubules is destabilizing and inhibits RPE autophagy. An essential function of RPE is the autophagic degradation of cellular debris and clearance of photoreceptor outer segment tips from the overlaying photoreceptor cells. Microtubule acetylation disrupts normal autophagosome movement as well as interactions between autophagosomes and lysosomes that are essential to RPE's autophagic activity. This leads to the accumulation of intracellular debris within the RPE, which could cause metabolic stress and also initiate drusen formation in the RPE. Microtubule acetylation also makes the RPE susceptible to complement-mediated mitochondrial injury, resulting in the formation of damaging aggregates of drusen. Thus, acetylated microtubule abundance in the RPE provides a measure of lipofuscin-induced injury and of the activation of the foregoing pathological cascade.

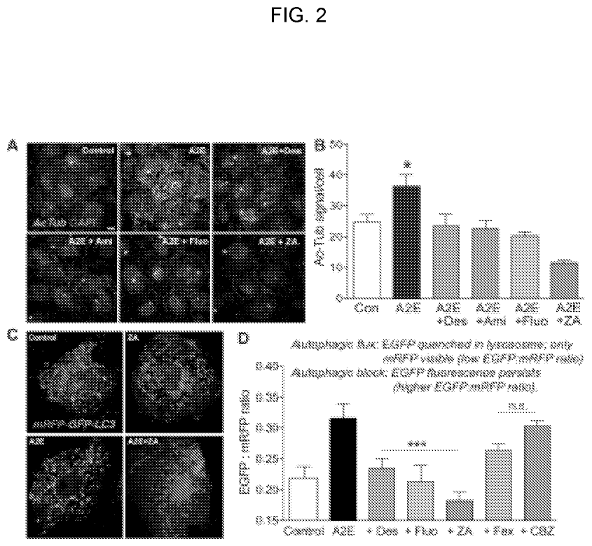

Primary RPE cultures treated with A2E had significantly increased acetylated tubulin compared to RPE cells that were not exposed to A2E, indicative of impaired autophagic capacity and resulting injury (FIG. 2B). Furthermore, application of ASM inhibitors subsequent to A2E treatment significantly reversed this A2E-induced effect. Among the tested ASM inhibitors, zoledronic acid provided the most potent reduction in acetylated tubulin abundance.

RPE cells treated with A2E, followed by ASM inhibitor application, were further evaluated for autophagic flux by live cell imaging. An autophagic reporter (tfLC3) comprising the autophagy target microtubule-associated protein 1A/1B-light chain 3 (LC3) fused with a tandem red fluorescent protein (mRFP) and enhanced green fluorescent protein (EGFP) was applied to the cultured RPE cells. Autophagosome uptake of tfLC3 and its delivery to lysosomes results in acidic quenching of the reporter's EGFP signal, thus simultaneous monitoring of mRFP and EGFP signals provides a means of observing and quantifying autophagosomy in RPE cells, wherein a reduction in the ratio of EGFP signal to mRFP signal is indicative of autophagic activity.

As depicted in FIG. 2D, EGFP:mRFP ratios were significantly elevated in A2E-treated RPE cells, relative to control cells that were not exposed to A2E, indicating impaired autophagy. Application of ASM inhibitors restored autophagy in A2E-treated cells. The most effective promoter of autophagy was zoledronic acid.

### Example 2. Development of Ultra Low Zoledronic Acid Doses

Based on the results described in Example 1, the in vivo effects of zoledronic acid on lipofuscin-induced injury of the RPE were assessed next. Zoledronic acid was administered to Abca4^{-/-} mice. Autosomal recessive Stargardt disease is a condition encompassing macular degeneration characterized by lipofuscin accumulation in the RPE. This form of Stargardt disease results from mutations in the ABCA4 gene. Therefore Abca4^{-/-} mice provide a model for the study of dry AMD, Stargardt disease, and other conditions wherein pathological lipofuscin accumulation occurs.

In an initial in vivo study, a single intraperitoneal injection of zoledronic acid was administered to the mice. The selected dose was 1 mg per kilogram body weight, or about 28 μg per mouse. This dose was selected based on the approved zoledronic acid dose used to treat osteoporosis and cancer in humans. While negative side effects are known in the use of zoledronic acid, the drug is generally considered to be well tolerated. However, observed significant deleterious effects were observed in mice when zoledronic acid was administered at 1 mg per kilogram body weight. Side effects included osteonecrosis of the jaw, problems feeding, and other symptoms such as weakness and lethargy.

Based on the foregoing results, the effects of administering much lower doses of zoledronic acid to Abca4^{-/-} mice were evaluated. Abca4^{-/-} mice were administered zoledronic acid in a single intraperitoneal injection at a dose of 5 μg per kg body weight, 100 times lower than the dose indicated for treatment of osteoporosis and cancer.

### Ceramide Accumulations

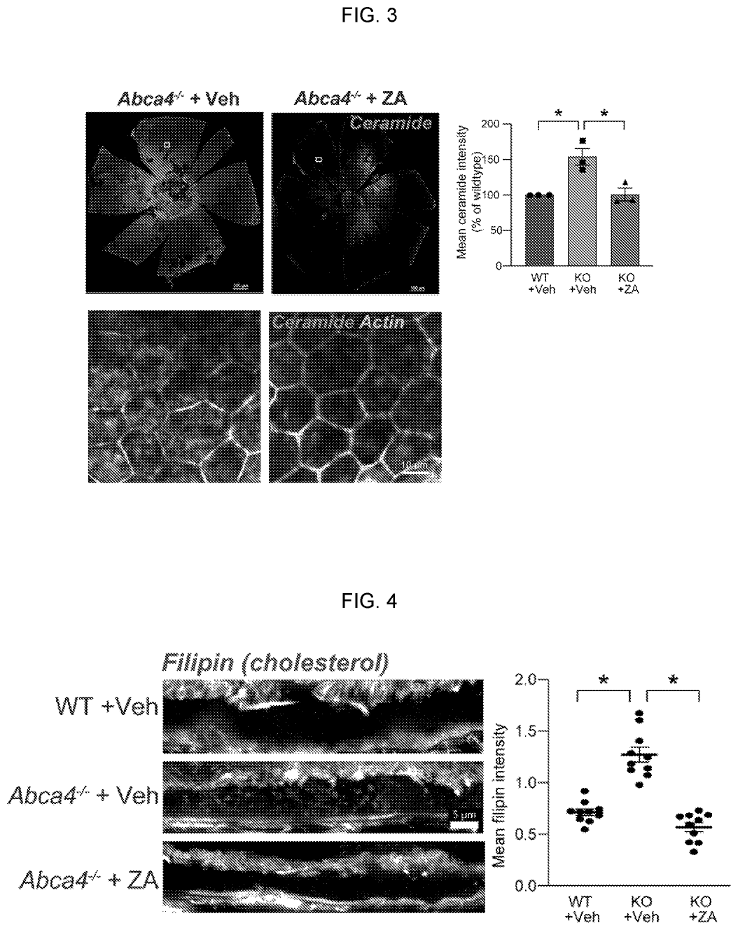

Sixteen-month-old wildtype and Abca4^{-/-} mice were administered vehicle or zoledronic acid (ZA, 5 μg/kg) by intraperitoneal injection (i.p.) three times/week for 8 weeks. At the end of the study, eyes were enucleated and the cornea, lens, vitreous, and retina were removed. 4-6 relaxing cuts were made to the eyecup to generate RPE flatmounts that were fixed in 4% paraformaldehyde. Ceramide levels were measured by immunostaining with a specific anti-ceramide antibody (Enzo) that has been validated extensively by us and other groups (Toops et al., 2015; Tan et al., 2016; Kaur et al., 2018; La Cunza et al., 2021). Images were captured on a Nikon spinning disc confocal microscope and ceramide fluorescence intensity per cell was quantified using Imaris (Bitplane). The results shown in FIG. 3 demonstrate that administration of low-dose zoledronic acid decreased ceramide accumulation.

### Cholesterol Accumulation

Twelve-month-old wildtype and Abca4^{-/-} mice were administered vehicle or zoledronic acid (ZA, 5 μg/kg) by intraperitoneal injection (i.p.) three times/week for 8 weeks. At the end of the study, the cornea, lens, vitreous were removed and the eyecups embedded for cryosectioning. 10 μm thick retinal cryosections were stained with filipin (Sigma), which specifically bind free cholesterol (Toops et al., 2015; Tan et al., 2016). The results depicted in FIG. 4 demonstrate that low-dose zoledronic acid decreased cholesterol accumulation in Abca4^{-/-} mice RPE

### Complement-Mediated Mitochondrial Fragmentation

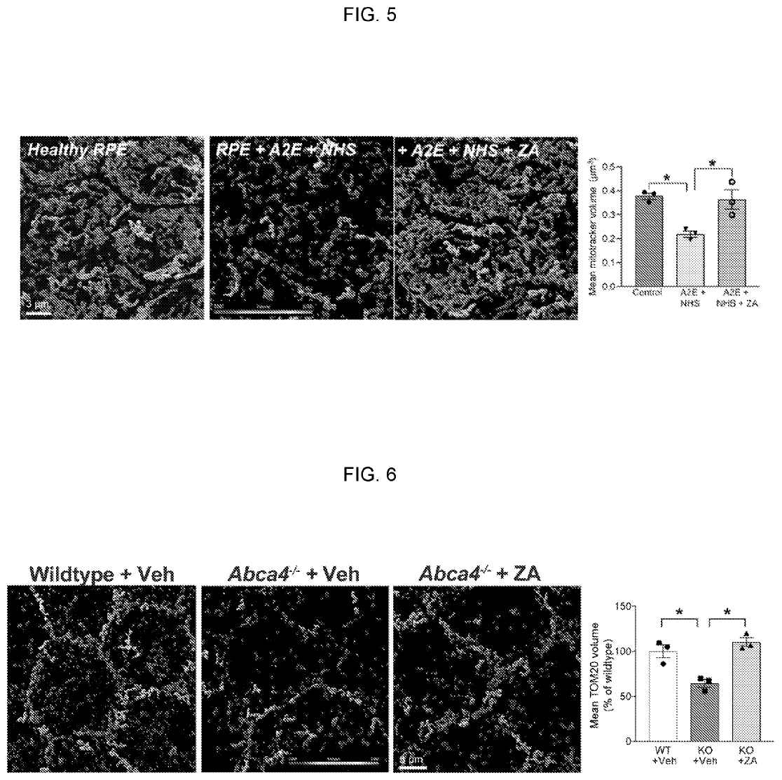

Highly differentiated polarized primary RPE cultures were established from freshly harvested porcine eyes as described (Toops et al., 2014). To induce complement-mediate mitochondrial injury, RPE monolayers were treated with the lipofuscin bisretinoid A2E, which compromises mechanisms that protect the RPE from complement activation (Tan et al., 2016). RPE cells were exposed to active complement components (10% normal human serum, NHS, for 10 min at 37° C.) to induce complement activation on the RPE cell surface. We showed previously that this results in mitochondrial fragmentation in the RPE (Tan et al., 2016; La Cunza et al., 2021). After NHS exposure, cells were treated with 1 μM zoledronic acid for 3 h. RPE mitochondria were labeled with MitoTracker Deep Red and cells were imaged live on a Nikon spinning disc confocal microscopy as we have described (Tan et al., 2016; La Cunza et al., 2021). Mitochondrial surfaces were reconstructed from 4-dimensional images using Imaris (Bitplane). The results depicted in FIG. 5 shows that zoledronic acid prevented complement-mediated mitochondrial fragmentation in the RPE in vitro.

Twelve-month-old wildtype and Abca4^{-/-} mice were administered vehicle or zoledronic acid (ZA, 5 μg/kg) by intraperitoneal injection (i.p.) three times/week for 8 weeks. At the end of the study, eyes were processed to generate RPE flatmounts (after removing the cornea, lens, vitreous, and retina) that were fixed in paraformaldehyde. Flatmounts were stained with an antibody to the outer mitochondrial membrane protein TOM20 to label mitochondria. Imaging and reconstruction of mitochondrial volumes were performed as in FIG. 5. The results depicted in FIG. 6 show that low-dose zoledronic acid prevents complement-mediated mitochondrial fragmentation in Abca4−/− mice RPE.

### Subretinal Microglia

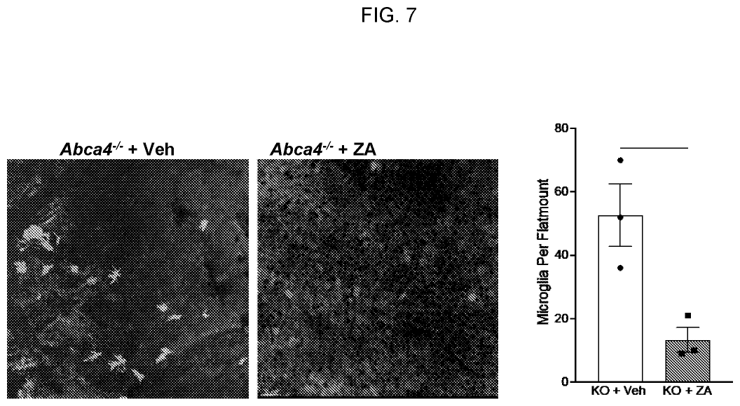

Sixteen-month-old wildtype and Abca4^{-/-} mice were administered vehicle or zoledronic acid (ZA, 5 μg/kg) by intraperitoneal injection (i.p.) three times/week for 8 weeks. At the end of the study, eyes were enucleated and the cornea, lens, vitreous, and retina were removed. 4-6 relaxing cuts were made to the eyecup to generate RPE flatmounts that were fixed in 4% paraformaldehyde. Flatmounts were immunostained with an antibody to ionized calcium-binding adaptor molecule 1 (Iba1), a protein expressed specifically by microglia. Images were captured on a Nikon spinning disc confocal microscope and microglial numbers per flatmount were quantified. The results depicted in FIG. 7 demonstrate that low-dose zoledronic acid decreased subretinal microglia in 18-month-old Abca4−/− mice.

### Photoreceptor Degeneration

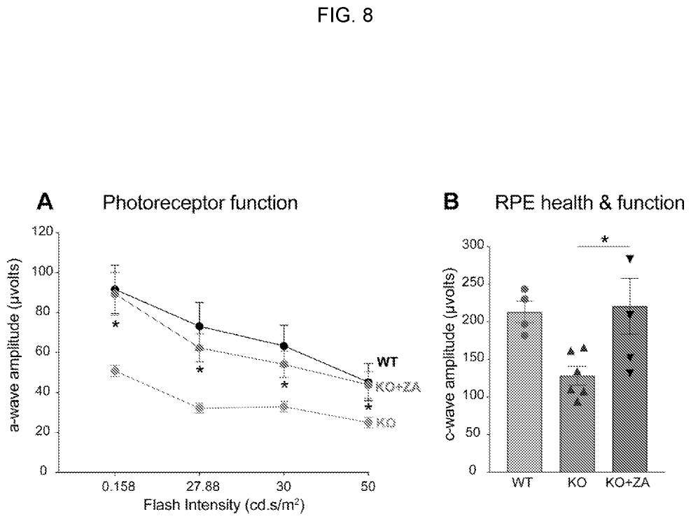

Changes in retinal function of 18-month-old wildtype and Abca4^{-/-} mice were determined by recording electroretinogram using the Celeris system (Diagnosys LLC, Lowell, MA) after vehicle or ZA administration for 8 weeks as above. The scotopic (dark-adapted) ERG responses were measured under flash intensities ranging from 0.158 to 50 cd·s/m². The a-wave amplitudes, which are a measure of photoreceptor function, were calculated and plotted as a function of flash intensities. The c-wave, which is a measure of the health and function of the RPE, was recorded under increasing flash intensities (0.158-100 cd·s/m²). The results depicted in FIG. 8 demonstrate that low-dose zoledronic acid prevented photoreceptor degeneration and visual deficits in 18-month-old Abca4−/− mice.

### Example 3. Treatment of RPE Cells with Adiporon In Vitro

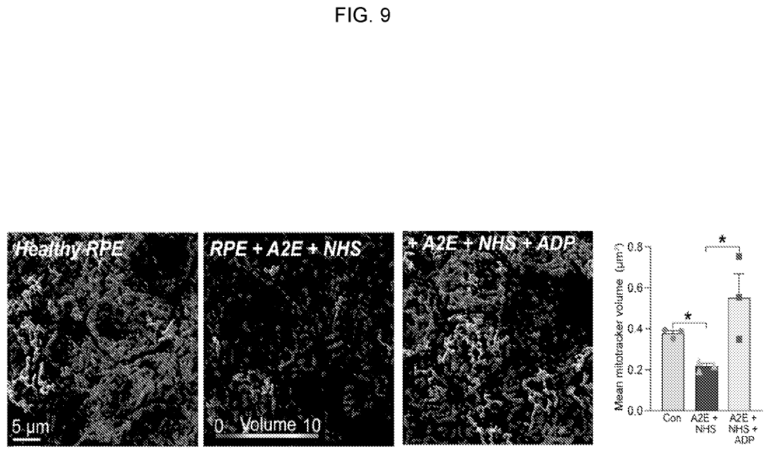

Highly differentiated polarized primary RPE cultures were established from freshly harvested porcine eyes as described (Toops et al., 2014). To induce complement-mediate mitochondrial injury, RPE monolayers were treated with the lipofuscin bisretinoid A2E, which compromises mechanisms that protect the RPE from complement activation (Tan et al., 2016). RPE cells were exposed to active complement components (10% normal human serum, NHS, for 10 min at 37° C.) to induce complement activation on the RPE cell surface. We showed previously that this results in mitochondrial fragmentation in the RPE (Tan et al., 2016; La Cunza et al., 2021). After NHS exposure, cells were treated with 1 μM adiporon (ADP) for 3 h. RPE mitochondria were labeled with MitoTracker Deep Red and cells were imaged live on a Nikon spinning disc confocal microscopy as we have described (Tan et al., 2016; La Cunza et al., 2021). Mitochondrial surfaces were reconstructed from 4-dimensional images using Imaris (Bitplane). The results depicted in FIG. 9 show that adiporon prevented complement-mediated mitochondrial fragmentation in the RPE in vitro.

Adiporon has been used at a dose of ˜25 μM for 24-48 h (10-50 μM range, with 25 μM being effective) in in vitro studies on hepatocytes, cardiomyocytes, pancreatic cancer cell lines, etc. The experimental results depicted in FIG. 9 were obtained using 1 μM Adiporon for 3 ours. We have also employed 0.01 μM (data now shown). These data shows that a 10-1000-fold lower dose (0.01-1 μM) for a shorter time (3 h) is effective in decreasing ceramide and preventing complement-induced mitochondrial injury in primary RPE cultures.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of treating a retinal disease in a subject in need of treatment therefor by administration to the subject of a therapeutically effective amount of a pharmaceutical composition comprising zoledronic acid or a derivative thereof, wherein the zoledronic acid or derivative is administered at an ultra-low dose.

2. The method of claim 1, wherein the retinal disease is a condition mediated by lipofuscin accumulation in RPE cells.

3. The method of claim 1, wherein the retinal disease is Stargardt macular dystrophy.

4. The method of claim 1, wherein the retinal disease is dry age-related macular degeneration.

5. The method of claim 1, wherein the retinal disease is selected from the group consisting of neuronal ceroid lipofuscinosis, Batten's Disease, Bietti's crystalline dystrophy, Niemann-Pick disease Type C, Doyne's honeycomb dystrophy, Farber disease, and Best vitelliform macular dystrophy.

6. The method of any of claims 1-5, wherein the pharmaceutical composition comprises or is incorporated within an implant; drug-eluting device, structure, or material; polymeric drug-eluting wafer; injectable hydrogel; or implantable hydrogel scaffold.

7. The method of any of claims 1-5, wherein zoledronic acid or the derivative is administered by intravitreal implant to deliver a dose of 50 ng/day to 50 μg/day to an eye.

8. The method of any of claims 1-5, wherein zoledronic acid or the derivative is administered as an eye drop solution or suspension, or ophthalmic ointment or gel at a dose of 50 ng/day to 50 μg/day to an eye.

9. The method of any of claims 1-5, wherein zoledronic acid or the derivative is administered by suprachoroidal injection at 50 ng/day to 50 μg/day to an eye.

10. The method of any of claims 1-5, wherein zoledronic acid or the derivative is administered systemically to provide a dose between 0.001 and 2.0 mg.

11. The method of any of claims 1-5, wherein zoledronic acid or the derivative is administered systemically to provide a dose between 0.01 and 2.0 mg.

12. The method of any of claims 1-5, wherein zoledronic acid or the derivative is administered systemically at a dose between 0.3 and 0.5 mg.

13. The method of any of claims 1-5, wherein zoledronic acid or the derivative is administered systemically at a dose between 0.3 and 0.5 mg is administered at a dose selected from the group consisting of 0.001 mg, 0.02 mg, 0.03 mg, 0.04 mg, 0.05 mg, 0.06 mg, 0.07 mg, 0.08 mg, 0.09 mg, 0.10 mg, 0.20 mg, 0.30 mg, 0.40 mg, 0.50 mg, 0.60 mg, 0.70 mg, 0.80 mg, 0.90 mg, and 1.0 mg.

14. The method of any of claims 1-5, wherein zoledronic acid or the derivative is administered systemically is administered at a dose between 100 ng to 10 g per kg body mass, at a dose between 1.0 and 7.0 μg per kg body mass, or at a dose of about 5.0 μg per kg body mass.

15. The method of any of claims 1-5, wherein zoledronic acid or the derivative is administered systemically in an amount of 0.1, 0.2, 0.3, 0.5, 1.0, 2.0, 3.0 4.0, 5.0, 6.0, 7.0, 8.0, 9.0, or 10.0 μg per kg body mass.

16. The method of any of claims 1-5, wherein the administration is by a route comprising any of intravenous delivery, intramuscular delivery, intraperitoneal delivery, or subcutaneous delivery.

17. The method of any of claims 1-16, wherein the pharmaceutical composition is administered at a frequency selected from the group consisting of: once per year, once per month, twice per month, weekly, twice weekly, every other day, daily, twice per day, and thrice per day.

18. The method of any of claims 1-17, wherein the pharmaceutical composition comprises zoledronic acid or a derivative thereof and any of an excipient, carrier, diluent, release formulation, drug delivery or drug targeting vehicle, and additional active therapeutic agent.

19. The method of claim 18, wherein the pharmaceutical composition comprises an adiponectin1 receptor agonist.

20. The method of claim 19, wherein the adiponectin1 receptor agonist comprises adiporon.

21. The method of any of claims 1-17, wherein zoledronic acid is co-administered with an adiponectin1 receptor agonist.

22. An intravitreal implant comprising zoledronic acid loaded with an amount of from 0.001 to 0.3 mg zoledronic acid.

23. The intravitreal implant of claim 22, wherein the implant is loaded with 0.005 to 2.5 mg zoledronic acid.

24. A topical ophthalmic preparation comprising 0.001-0.05 mg/dose of zoledronic acid.

25. A topical ophthalmic preparation of claim 24, comprising 0.005-0.05 mg/dose of zoledronic acid.

26. An injectable ophthalmic preparation comprising zoledronic acid at a concentration of 1 μg/ml-10 mg/ml.

27. The injectable ophthalmic preparation of claim 26, comprising zoledronic acid at a concentration of from 10 μg/ml-1 mg/ml.

28. The injectable ophthalmic preparation of claim 26 or 27, wherein the ophthalmic preparation comprises zoledronic acid conjugated to dendrimers or formulated as nano particles.

29. A method of treating a retinal disease in a subject in need of treatment therefor comprising administration to the subject a therapeutically effective amount of a pharmaceutical composition comprising adiporon to the subject.

30. A method of treating a retinal disease in a subject in need of treatment therefor by administration to the subject of a therapeutically effective amount of a pharmaceutical composition comprising a bisphosphonate ASM/FDPS inhibitor, wherein the bisphosphonate ASM/FDPS inhibitor is administered at an ultra-low dose.

31. The method of claim 29 or 30, wherein the retinal disease is a condition mediated by lipofuscin accumulation in RPE cells.

32. The method of claim 29 or 30, wherein the retinal disease is Stargardt macular dystrophy.

33. The method of claim 29 or 30, wherein the retinal disease is dry age-related macular degeneration.

34. The method of claim 29 or 30, wherein the retinal disease is selected from the group consisting of neuronal ceroid lipofuscinosis, Batten's Disease, Bietti's crystalline dystrophy, Niemann-Pick disease Type C, Doyne's honeycomb dystrophy, Farber disease, and Best vitelliform macular dystrophy.

35. The method of any one of claims 29-34, wherein adiporon or the bisphosphonate ASM/FDPS inhibitor wherein is administered at a dose between 0.001 and 2.0 mg, at a dose between 0.1 and 1.0 mg, or at a dose between 0.3 and 0.5 mg.

36. The method of any one of claims 29-34, wherein adiporon or the bisphosphonate ASM/FDPS inhibitor is administered at a dose selected from the group consisting of 0.001 mg, 0.002 mg, 0.003 mg, 0.004 mg, 0.005 mg, 0.006 mg, 0.007 mg, 0.008, mg, 0.009 mg, 0.01, 0.02 mg, 0.03 mg, 0.04 mg, 0.05 mg, 0.06 mg, 0.07 mg, 0.08 mg, 0.09 mg, 0.10 mg, 0.20 mg, 0.30 mg, 0.40 mg, 0.50 mg, 0.60 mg, 0.70 mg, 0.80 mg, 0.90 mg, and 1.0 mg.

37. The method of any one of claims 29-34, wherein adiporon or the bisphosphonate ASM/FDPS inhibitor is administered at a dose between 100 ng to 10 μg per kg body mass, at a dose of between 1.0 and 7.0 μg per kg body mass, or at a dose of 5.0 μg per kg body mass.

38. The method of any one of claims 29-34, wherein adiporon or the bisphosphonate ASM/FDPS inhibitor is administered at a dose selected from the group consisting of 0.1, 0.2, 0.3, 0.5, 1.0, 2.0, 3.0 4.0, 5.0, 6.0, 7.0, 8.0, 9.0, and 10.0, μg per kg body mass.

39. The method of any of claims 29-38, wherein the pharmaceutical composition is administered at a frequency selected from the group consisting of: once per year, once per month, twice per month, weekly, twice weekly, every other day, daily, twice per day, and thrice per day.

40. The method of any of claims 29-39, wherein administration is by a route comprising any of: systemic delivery; local delivery; intravenous delivery; intramuscular delivery; intraperitoneal delivery; topical delivery; subcutaneous delivery; intraocular delivery; and topical delivery to the eye.

41. The method of any of claims 29-39, wherein the pharmaceutical composition comprises one or more bisphosphonate ASM/FDPS inhibitors or adiporon and any of an excipient, carrier, diluent, release formulation, drug delivery or drug targeting vehicle, and additional active therapeutic agent.
